(19) United States
(12) Patent Application Publication
(10) Pub. No.: US 2013/0066349 A1
(43) Pub. Date: Mar. 14, 2013

(54) STIMULATED VOC CHARACTERIZATION

(71) Applicants: Richard Lee Fink, Austin, TX (US); Zvi Yaniv, Austin, TX (US); Leif Thuesen, Austin, TX (US); Alexei Tikhonski, Cedar Park, TX (US); Royce W. Johnson, Universal City, TX (US)

(72) Inventors: Richard Lee Fink, Austin, TX (US); Zvi Yaniv, Austin, TX (US); Leif Thuesen, Austin, TX (US); Alexei Tikhonski, Cedar Park, TX (US); Royce W. Johnson, Universal City, TX (US)

(73) Assignee: Applied Nanotech Holdings, Inc., Austin, TX (US)

(21) Appl. No.: 13/611,864

(22) Filed: Sep. 12, 2012

Related U.S. Application Data
(60) Provisional application No. 61/534,025, filed on Sep. 13, 2011.
(60) Provisional application No. 61/583,288, filed on Jan. 5, 2012.

(51) Int. Cl. A61B 17/32

(52) U.S. Cl. 606/169

(57) ABSTRACT

An electronic odor sensor is used in conjunction with a surgical tool, for example when wounds are cleansed to remove dead tissue and exudates, known clinically as debridement. The surgical tool will atomize substrate tissues and thereby mechanically generate vapors that can be sensed. Abrasion will likewise atomize substrate tissues liberating odors. Air near the surgical tool is collected and fed into the electronic odor sensor. The odor is analyzed by the sensor and a signal fed back based on the analysis.

This application claims priority to U.S. Provisional Patent Applications Ser. Nos. 61/534,025 and 61/583,288, which are hereby incorporated by reference herein.

### TECHNICAL FIELD

The present invention relates to the use of sensing particles in gases, such as odors and/or volatile organic compounds (“VOCs”), and creating a feedback mechanism for various uses and applications.

### BACKGROUND AND SUMMARY

There are many areas in which information feedback from gas analysis can be used for quality control and identification of persons, diseases, dangerous industrial and explosive materials, and monitoring human health conditions. Such use of odor and/or VOC detection can assist in quality control in manufacturing materials, including drugs and pharmaceuticals. As an example, Johnson & Johnson was required to recall medications as a result of chemical contamination that resulted in an off-odor to the medication. Odor feedback during the manufacturing process would have detected the presence of the odor early in the manufacturing cycle, thereby eliminating costly waste and assisting to maintain product integrity.

Another example is the use of odor and/or VOC detection for uniquely identifying an individual. This may be used at various points of entry and/or security check points. This may be used on various means of transportation (e.g., planes, ships and boats, buses, trains and automobiles). An example of the use of the technology for uniquely identifying an individual is by monitoring specific VOCs that are known to be genetically controlled. Examples of these VOCs include but are not limited to 3-methybutanal, 2-pentanone, Z-3-methyl-2-hexenoic acid, E-3-methyl-2-hexenoic acid, 7-octenoic acid, 3-hydroxy-3-methyl-hexanoic acid, 3-methybutyric acid, 2-methybutric acid, p-cresol, phenol, phenylacetic acid, octanal, nonanal, and decanal.

Another example is the use of odor and/or VOC detection for identifying a classification of an individual. This classification may be race, gender, family, tribe, and/or membership in specific communities.

Yet another example is the use of odor and/or VOC detection for determining if an individual or individuals have been exposed to a general class of materials, such as explosives or dangerous chemicals (e.g., nerve agents or other materials known as Chemical and Biological Warfare Agent threats).

A further example is the use of odor and/or VOC detection for identifying and/or classifying the relationship of an individual to an object. This may be used for forensic purposes, (e.g., providing information related to who may have worn clothing found at a crime scene).

Another example is the use of odor and/or VOC detection for monitoring human, animal, or plant health. The use of odor and/or VOC detection can be utilized to identify specific diseases in a person, such as, but not limited to, lung cancer or other cancers, diabetes, emphysema, or asthma.

Another example is the use of odor and/or VOC detection for promoting or improving marketing of products. An example may be to maintain a certain aroma in a room or area that would promote sales. The system may be a feedback loop between an odor and/or VOC producing mechanism and a sensor that monitors the amount of odor in a room or area, in order to maintain the odor and/or VOC level within a safe and desirable range. The intensity of the odor and/or VOC concentration may be varied from day-to-day or time of day (e.g., coffee in the morning, bacon at lunch, etc.). The type of odor or VOC may be selected to promote the marketing of certain produce (e.g., fresh tomatoes), or even of new homes or other real-estate (e.g., fresh baked cookie odor or vanilla odor).

A further example is the use of odor and/or VOC detection for theft control.

Another example is the use of odor and/or VOC detection for monitoring or controlling an industrial process, such as but not limited to doping an odorant into a gas stream.

Active infections have long been known to exude noxious odors. For at least the prior decade, the use of e-nose technologies (electronic odor sensors) have been proposed as a method of monitoring infected wounds. Such use of electronic odor sensors has not been successful because an instrument was not available to detect the foul odors. And for low level infections or for mere colonizations of bacteria in wounds, there were not enough of the VOCs (volatile organic compounds) produced for electronic odor sensors, nor human noses, to detect them. In the case of sessile bacteria, typically embedded in a biofilm, or pathogens present only in spore form, the VOCs of active bacterial metabolism may not be present at all.

Electronic odor sensors require a vaporous sample to process. Without VOCs available, even a maximally sensitive electronic odor sensor will fail to detect the bacteria. It is now well known that chronic wounds are caused by bacteria embedded in biofilms of their own manufacture (James, G. A. “Biofilms in chronic wounds,” Wound Rep Regen, 2007). It is the sessile and/or quiescent pathogens in wound beds that drive the chronicity of intransigent wounds and therefore of importance for achieving lower costs and improving outcomes in wound care. The biofilm protects the bacteria from endogenous and exogenous antimicrobial attack, but also limits the metabolic activity of the bacteria. This screens the presence of the bacteria from normal detection by their emitted odors or fluids. What is needed is a mechanism that will liberate characteristic VOC signatures for positive detection of the presence of these bacteria or their vegetative byproducts.

A solution is to apply a physical or chemical challenge to the tissues in which the suspected bacteria are contained. Such a challenge will stimulate, excite, liberate, or generate VOCs that are detected by an electronic odor sensor (also referred to herein as an “e-nose”). In the same way that crushing a mint leaf releases its scent, performing interventions on wounds will produce odors that can be electronically analyzed.

More specifically, wounds are cleansed to remove dead tissue and exudates, known clinically as debridement. The cleansing process may involve blades, abrading devices, chemical cleansers, or advanced chemo-mechanical cleaning such as with ultrasound or plasmas. Blades will atomize substrate tissues and thereby mechanically generate vapors that can be sensed. Abrasion will likewise atomize substrate tissues liberating odors. Chemical cleansers cause reactions in the tissue surface, generally oxidations, which will produce signature vapors. And the advanced cleansing modalities will act in both ways to produce signature vapors.

Embodiments of the present invention may be in the form of a sampling port attached to the wound care site or tool in use to perform the intervention(s). In the case of a debridement blade, a sampling line may be integrated in the scalpel handle. In the case of cleansers, a sampling line may be attached to the patient's skin in the near vicinity of the wound, or to cleanser-dispensing applicators. In the case of advanced debridement modalities, they may involve an applicator handle similar to blade handles, and also include cabling or plumbing that routes to and from an active console that contains the generators of plasma, ultrasound, water mist, etc. Such cabling/plumbing would allow for easy incorporation of a sampling system for odor analysis.

It is also important to note that the application of energy or chemicals to tissues may create new chemical signature vapors, i.e., vapors not natively present in the targeted tissues. These new components can be uniquely specific to the targeted condition. For example, with application of peroxide cleansers, the free-radical reactions with fatty acids and lipopolysaccharides of biofilms will generate oxidized small molecules that are indicators of the presence of biofilms.

An aspect of this disclosure is the use of odor and/or VOC detection for monitoring the progress of cutting or ablation mechanisms used to remove damaged, diseased and/or dead human matter such as flesh or bone matter. For example, this tool may be used to monitor the progress of removing or treating tissue using a debridement tool. By monitoring the odors resulting from the debridement process, one can determine when the damaged tissue has been removed or that the tool is removing healthy tissue (i.e., a sort of end-point detector).

### BRIEF DESCRIPTION OF DRAWINGS

FIG. 1 illustrates an example of an e-nose used by a surgeon in conjunction with a scalpel.

FIG. 2 illustrates an example of an e-nose used by a surgeon in conjunction with a debridement tool.

FIG. 3 illustrates a block diagram of a trap—GC-DMS system.

FIG. 4 illustrates air sampled by pumping through a trap.

FIG. 5 illustrates the trap of FIG. 4 releasing the sample into the GC column by heating and the sample flows through the column.

FIG. 6 illustrates the GC column heated allowing the sample components to separate while flowing through the column and into the DMS filter where the sample is analyzed; the system may be cleaned with filters in an air recirculation system.

FIGS. 7A-7B illustrates a DMS on filter used to separate different analytes in a gas sample.

FIGS. 8A-8B show an example of an ionization source coupled to a DMS analyzer.

### DETAILED DESCRIPTION

Herein, the terms “air,” “gas,” and “vapor” are used interchangeably to refer to the volume of gas containing analytes (particles) sensed by an e-nose device. Additionally, the e-nose may be used to detect such analytes (particles) dissolved in a liquid fluid.

For embodiments disclosed herein, levels of detection may be in a percent concentration range (e.g., breath analysis for measuring breath alcohol levels), or down to very minute levels such as parts per trillion (e.g., for disease detection or uniquely identifying an individual or industrial process control).

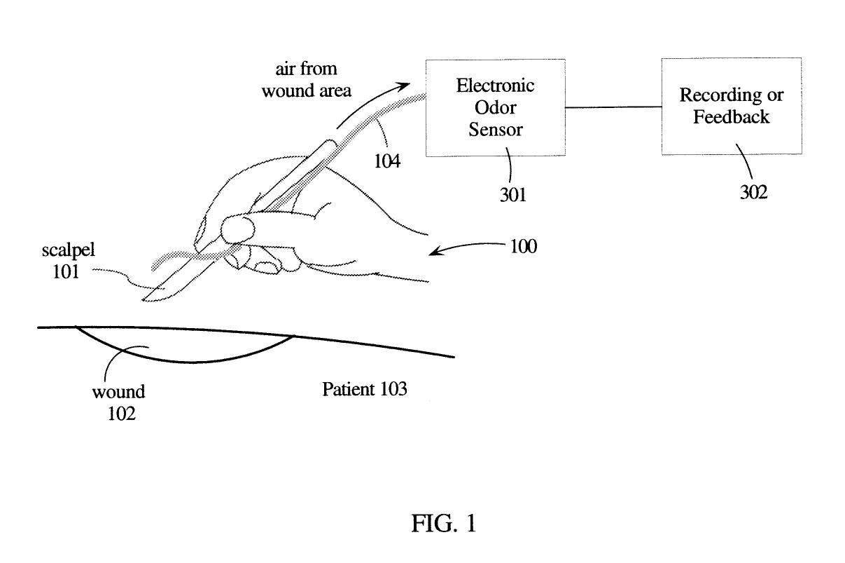

FIG. 1 illustrates an example of an electronic odor sensor 301 used by a person (e.g., a surgeon) in conjunction with a scalpel 101. The surgeon 100 is holding a scalpel 101 that also has a tube 104 connected to the scalpel 101 or the hand 100 such that an open end of the tube 104 is near the blade of the scalpel 101. Air (a gas) is sucked (e.g., by a pump, not shown for the sake of simplicity) into the tube 104 from the end of the tube 104 near the scalpel blade 101 such that the air is collected near the scalpel blade 101 and fed into the electronic odor sensor 301. The odor (e.g., particles in the air or gas) is analyzed by the sensor 301 and/or a computer, 302 coupled to the sensor, and may be digitally recorded. In another example, the odor is analyzed and then a signal(s) is fed back to the surgeon based on the analysis (e.g., an amount of a certain odor signature that may be important for the surgeon 100 in a procedure for the care of the wound 102 of a patient 103). It is possible to both record and provide feedback at the same time. An example of an odor liberated and analyzed for feedback would be the volatile components of bacterial biofilm embedded in a wound bed and released during debridement, wherein a measured threshold amount of a specified odor (indicating the existence of the bacterial biofilm in the wound) produces an audible alert to the surgeon informing the surgeon whether to continue to debride the wound. Other clinically relevant tissues and their odors to target for release, analysis, and feedback to clinicians would include cancerous lesions and local colonies of bacteria or viruses. Examples of odors from cultures of bacteria include, but are not limited to, acetaldehyde acetic acid, ethanol, acetone, ammonia, methyldisulfide, dimethylsulfide, dimethyldisulfide, 2-aminoacetophenone, and 2-propanol. These are examples of compounds that may be detected directly in the sensor. A debridement tool may also cause chemical reactions that create other compounds that are detected, such as methane combustion products, oxidized fatty acids, or hydrogen sulfide (H₂S).

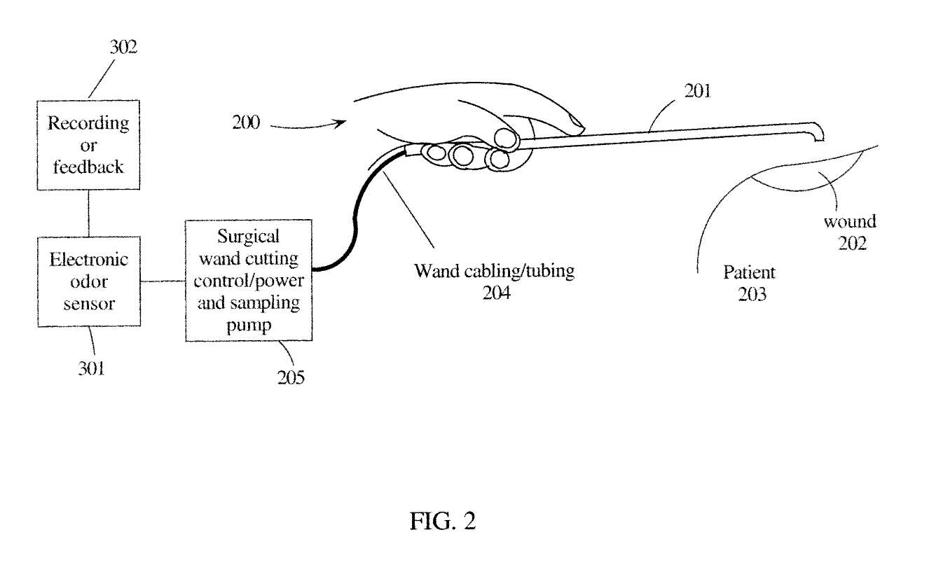

FIG. 2 illustrates an example of an electronic odor sensor 301 used by a person (e.g., surgeon) in conjunction with a debridement tool 201 (e.g., uses plasma, ultrasound, water mist, or electrosurgical action). In this case, cabling from the wand controller 205 may include cabling/tubing 204 that collects air, gas, or vapor near the tip of the wand 201. The tubing 204 may also collect fluid that contains dissolved gases that may be of interest to the surgeon 200. To use the wand 201 as part of an odor sensor, the tip of the wand 201 may be brought close to the wound 202 or touches the wound 202 of the patient 203. The active mechanism of the wand 201 (e.g., plasma, ultrasound, etc.) may be on or off, or may be activated at a low or intermediate power below a threshold needed for cutting or debriding of tissue but sufficient enough for challenging the wound 202 to produce VOCs for sensing by the e-nose 301 that help the surgeon 200 characterize the wound tissue 202, or bacteria that may be located in the wound 202. In a similar manner as described with respect to FIG. 1, a feedback mechanism 302 may be utilized by the surgeon 200.

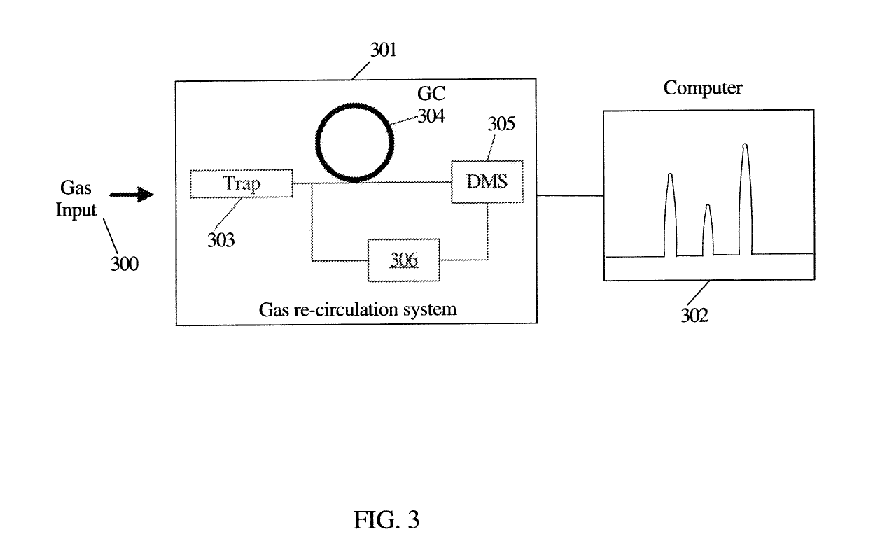

FIG. 3 illustrates a simplified block diagram of an electronic odor sensor 301 for sensing VOCs that are liberated by wound care cleaning and/or debridement processes as described with respect to FIGS. 1 and 2. In the system 301-302, a gas chromatograph (GC) 304 may be coupled with a differential ion mobility spectrometer (DMS) 305, the combination also referred to as GC/DMS. Input gas 300 comes into the e-nose 301 through a port. In a configuration of the e-nose 301, the input gas 300 is passed through a trap 303 that concentrates the VOC analytes in the gas. Then the concentrated gas is passed through a GC column 304. The GC column 304 is then eluted into the differential mobility spectrometer (DMS) 305. The DMS 305 is part of a family of ion mobility spectrometers that is related to High-Field Asymmetric Waveform Ion Mobility Spectrometry (FAIMS) (see, e.g., Roger Guevremont, “High-Field Asymmetric Waveform Ion Mobility Spectrometry,” Canadian J. of Anal. Sciences and Spectroscopy, Vol. 49(3), pp. 105-113, 2004, which is hereby incorporated by reference herein). Examples of tools that may be used to monitor VOCs are gas chromatographs, gas chromatographs coupled to mass spectrometers, and gas chromatographs coupled to ion mobility spectrometers. Ion mobility spectrometers may include time-of-flight spectrometers and FAIMS (Field Asymmetric Waveform Ion Mobility Spectrometry). In some cases, the mass spectrometer and/or the ion mobility spectrometer may be used independent of a gas chromatograph. In some cases, the mass spectrometer may be coupled with an ion mobility spectrometer. In some cases, a gas chromatograph may be coupled to both an ion mobility spectrometer and a mass spectrometer, either in series or in parallel.

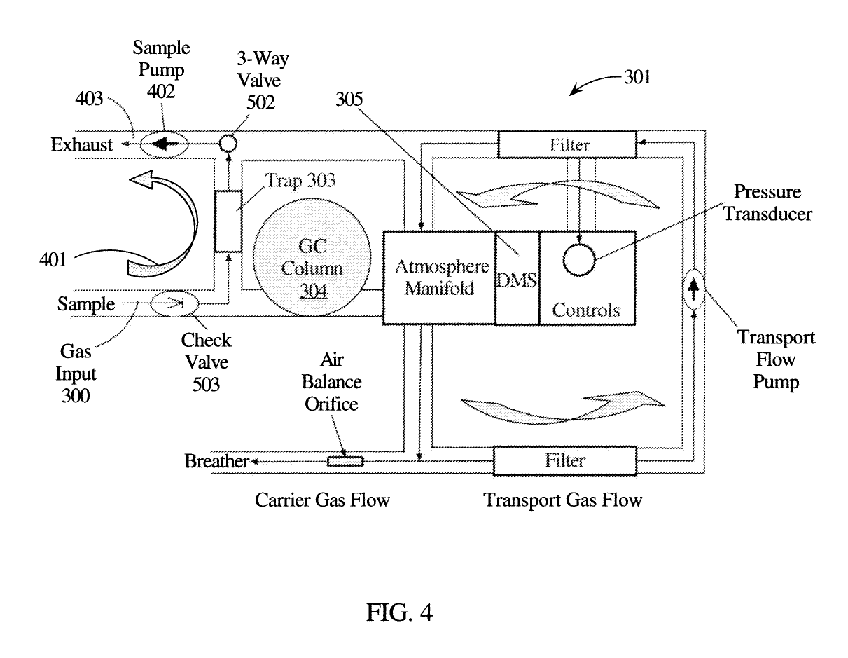

FIGS. 4-6 illustrate an operation of the e-nose 301 in more detail. A first step is trap loading. The left side of the diagram in FIG. 4 shows the system drawing in the gas 300 (e.g., from tube 104 or 204-205) into the sample port as shown by arrow 401. The gas 300 is pumped through the trap 303 where the analytes in the gas 300 are concentrated during several seconds of collection. A pump 402 may be used to help transport the gas 300 through the trap 303. Gas that passes through the trap 303 may then be exhausted through a port 403.

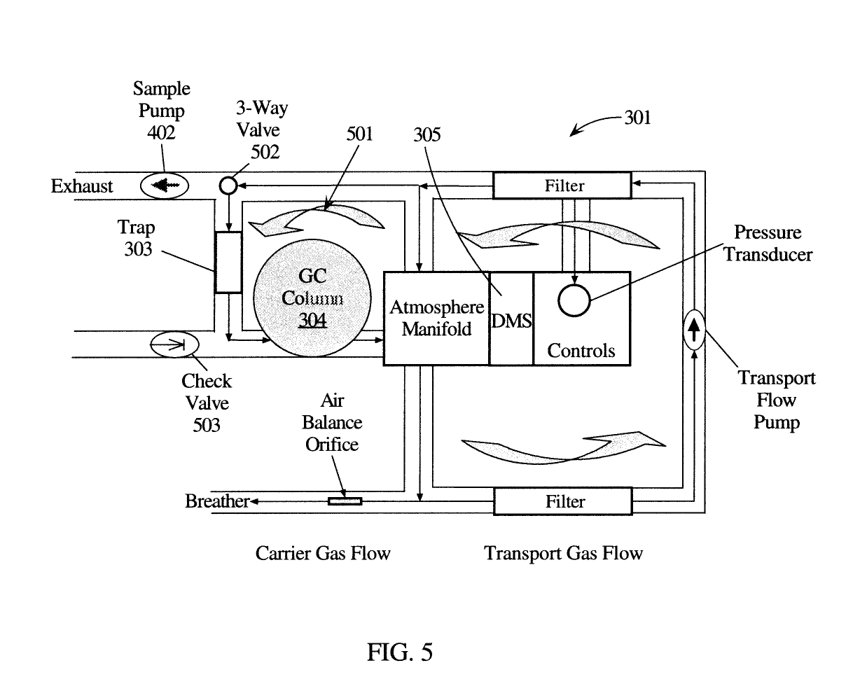

Referring to FIG. 5, a next step involves releasing the analytes that are concentrated in the trap 303 into the GC column 304. This may be performed by closing the flow of gas 300 shown by arrow 401 through the trap 303 from the sampling port and opening the valve to the gas in the recirculating loop as depicted by arrow 501. A three-way valve 502 may be activated to begin flow 501. A check valve 503 may be used to keep this gas flow from escaping from the sample port. Other alternative configurations may be used. When gas flow 501 starts to flow through the trap 303, the trap 303 may be heated to release the analytes into the GC column 304. Analytes are carried through the GC column 304 at a low flow rate (see arrow 501 over the GC column 304). The GC column 304 may separate components of the analytes while maybe adding a time dimension to the data, which enhances an ability to identify the chemicals of interest.

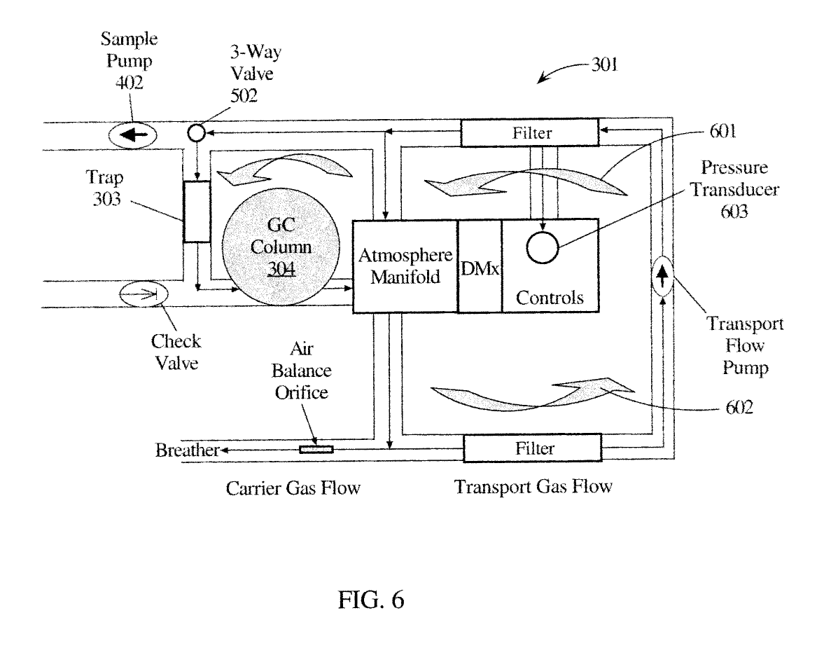

Referring to FIG. 6, the analytes are eluted from the GC column 304 into the main recirculation flow of the DMS part of the e-nose 301 (see arrows 601-602 near pressure transducer 603). This is where the ionization and analysis of the sample occurs. The trap 303 may go through a cooling cycle at this time. Depending on the analyte(s) and the configuration of the e-nose 301 (e.g., type and length of GC column, etc.), the analytes may take approximately 10-1200 seconds to elute from the GC 304, but only spend a small fraction of a second passing through the DMS 305 because of the higher flow rate through the DMS and smaller distance travelled (e.g., 1-2 cm). Flow rates through the GC column may be 1-5 sccm (standard cubic centimeter per minute), and flow rates through the DMS may range from 100-1000 sccm. The length of the GC column may be 0.01-20 meters. A shorter length GC column made up of arrays of capillary tubes in parallel may be utilized.

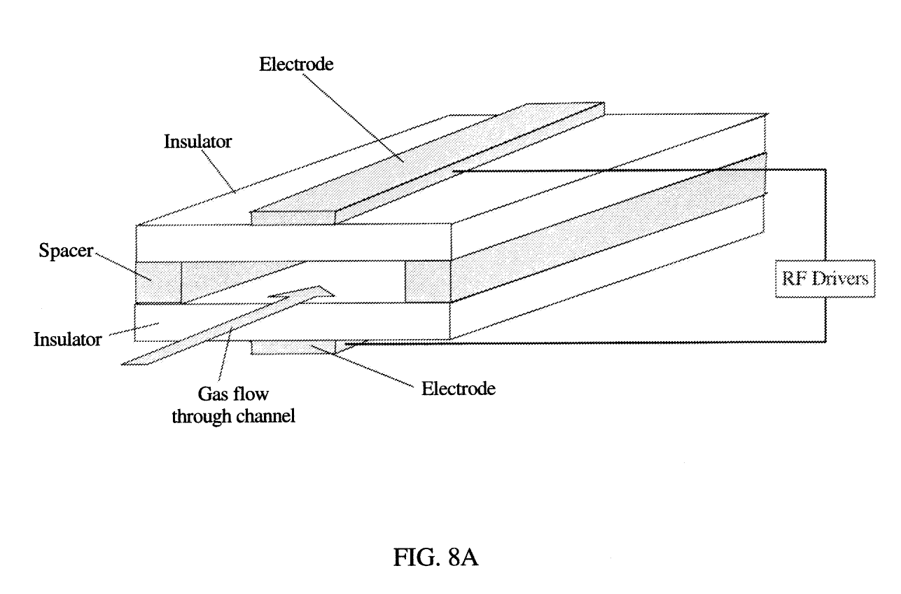

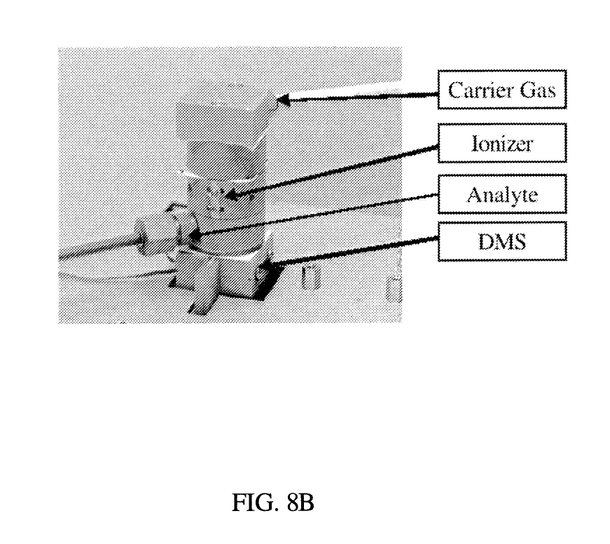

The VOC analyte molecules are ionized as they enter the DMS 305. One technique used to create gas ions is to place a radioactive source material 701 (either beta emitter or alpha emitter) next to the gas flow 601 and 602. Alternatively, an ion generator that does not utilize radioactive sources may be utilized (see FIGS. 8A-8B).

The gas sample is separated by the DMS filter 305 to further improve the analyte identification. By carefully calibrating the tool for the chemicals of interest, the analyte concentrations in the gas sample are known. Once the analytes and their concentrations have been identified, the data is further analyzed for the desired purpose. In another analysis method, the identification details may be compared to a previously determined database of compounds and concentration ratios seen with known disease conditions to determine disease status. In the case of wound debridement, this would be conditions of infection and biofilm presence as determined to be present by standard methods and clinical experts. Alternative analyses of compounds identified may be performed by pattern recognition methods such as principal component mapping, k-nearest neighbor classification, or neural network recognition. The analyte identification step may be alternatively bypassed and the system simply map disease conditions to the signal output of the DMS filter. This has an advantage of not requiring detailed calibration of the tool for specific chemical identifications, but produces no intermediate information for verification of the biochemical identity of the targeted disease condition.

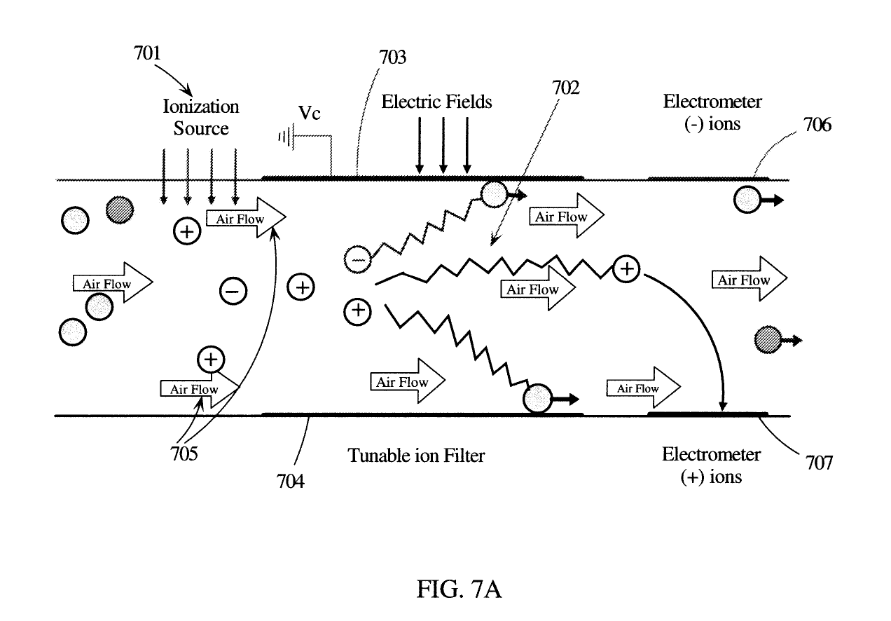

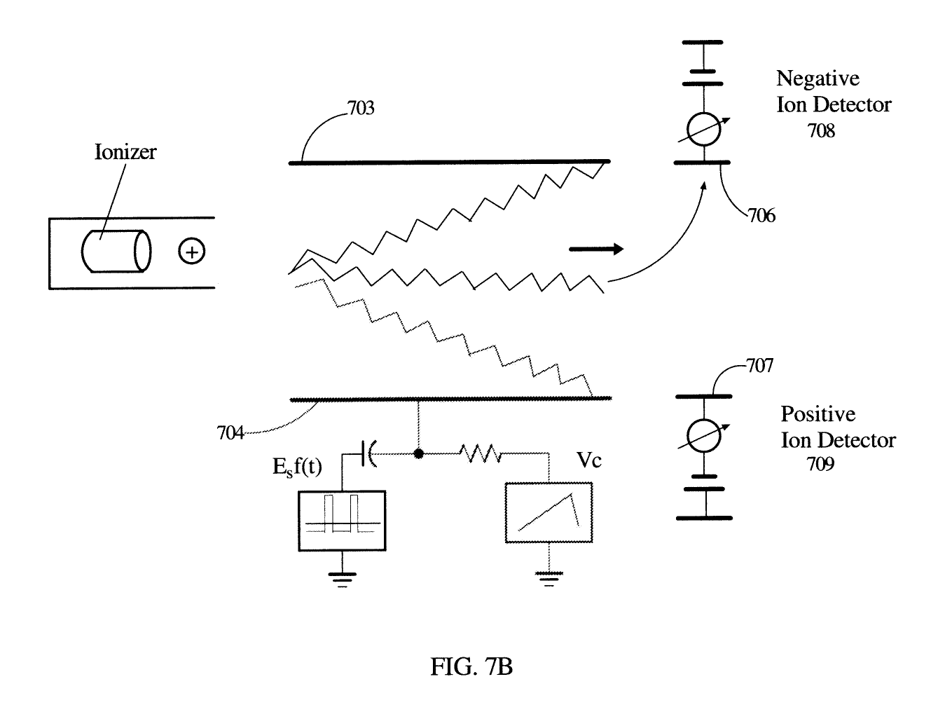

The DMS is essentially an ion filter operating in a gas environment. The gas environment may be filtered and dried (de-humidified) air at near atmospheric pressure. Other gasses may be used such as high purity nitrogen, argon or other noble gasses. A principle of operation of the DMS is illustrated in FIGS. 7A-7B, and as disclosed in U.S. published patent applications nos. 2012/0160997 and 2010/0127167, which are both hereby incorporated by reference herein.

As stated previously, DMS is one of a family of Ion Mobility Spectrometry (IMS) tools that has several advantages compared to standard time-of-flight IMS approaches. Mainly it provides a richer set of data and improves on the chemical selectivity while maintaining sensitivity. Gas chromatography coupled with differential mobility spectrometry (GC/DMS) has a number of advantages.
GC/DMS has the sensitivity and fidelity to detect and measure a wide variety of compounds at very low concentration levels (ppb is common).
GC/DMS can be significantly miniaturized because it does not require a vacuum to operate.
GC/DMS can be made low-cost compared to GC/mass spectrometry.
GC/DMS tools typically require radioactive gas ion sources but disclosed herein is a non-radioactive gas ionization source that will significantly decrease the cost of ownership and administrative burden.

A physical principle of DMS is based upon the relationship of an ion's velocity in a gas being proportional to an applied electric field strength, or

\vec{v}ᵢ(\vec{E})=kᵢ(\vec{E})\vec{E}

where kᵢ(E) is the ion mobility.

kᵢ(E) depends on the carrier gas pressure, composition and temperature as well, but those variables can be fixed by design. The DMS takes advantage of the non-constant and non-linear electric field dependence of the ion mobility. Referring to FIGS. 7A-7B, a scheme that a DMS employs places an asymmetric RF electric field in the ion drift region 702. The electric field may be generated by voltages placed on electrodes 703 and 704 that contain the gas flow in the ion drift region 702 illustrated in the cross-section view FIG. 7A. These electrodes may be 2-10 mm wide and 10-30 mm long in the direction of the air flow 705. The gap between the electrodes may be 0.1-1.0 mm. Two other non-conducting walls (not shown) may complete the form of the channel of the drift region 702. An electric waveform may be created by placing voltages on the electrodes 703 and 704. The waveform cycle may alternate between a high field, short pulse duration and a lower field, longer pulse duration of the opposite polarity such that the total integrated area of a cycle is zero. The non-linear mobility results in ions having a net non-zero drift velocity in the y direction so they eventually strike the RF electrodes. The superposition of a weak DC electric field in the y direction cancels the RF induced net drift and the ions then pass through the filter, where they are collected onto electrodes 706 (e.g., DC biased positive) and 707 (e.g., DC biased negative), and the ion current becomes the detected signal (e.g., as measured by high sensitive current meters 708 and 709). By sweeping the weak DC electric field successive ion components pass through the ion filter and are selectively measured. Concepts of exemplary DMS are described in detail in Kolakowski B. M., Mester Z., “Review of applications of high-field asymmetric waveform ion mobility spectrometry (FAIMS) and differential mobility spectrometry (DMS),” Analyst 132 (9), pp. 842-64, September 2007; I. A., Buryakov, E. V. Krylov, E. G. Nazarov U. Kh. Rasulev, “A new method of separation of multi-atomic ions by mobility at atmospheric pressure using a high-frequency amplitude-asymmetric strong electric field,” International Journal of Mass Spectrometry and Ion Process, Vol. 128, pp. 143-148, 1993; and E. Krylov, E. G. Nazarov, R. A. Miller, B Tadjikov, and G. A. Eiceman, “Field Dependence of Mobilities for Gas-Phase-Protonated Monomers and Proton-Bound Dimers of Ketones Planar PFAIMS,” J. Phys Chem. A 2002, Vol. 106, pp. 5437-5444, which are all incorporated by reference herein.

Another electronic odor sensor embodiment is Ion Mobility Spectrometry (“IMS”). IMS is similar to DMS with a difference. IMS essentially uses a time-of-flight (“TOF”) measurement to measure how fast a given ion is able to move through a uniform electric field at a given pressure/atmosphere. DMS does not rely on a TOF, but instead uses the differences in mobility of different ions to detect only the ion as it passes through an ion filter.

Another electronic odor sensor embodiment is gas chromatograph mass spectrometry (“GC/MS”). GC/MS systems are sensitive and capable of identifying the constituents of a large number of unique combinations of VOCs signatures to diagnose many different types of diseases. However, a drawback to this technology is in the lengthy operating times, cost, and size. GC/MS systems may cost approximately $75K-$150K. In addition, GC/MS systems require vacuum pumps, which may limit miniaturization and increase power consumption. In contrast, DMS technology has advantages for portable applications and can achieve the required sensitivity.

Another electronic odor sensor embodiment is a quartz microbalance (“QBM”). This technology relies on the change in resonant frequency of a micromachined quartz beam when the molecules of a desired analyte adsorb onto it, thus changing the resonant mass. These beams are patterned with special coatings, such as metalloporphyrin complexes to selectively capture molecules of interest. To detect multiple analytes simultaneously, arrays of these sensors are used, making it more difficult to fabricate as well as implement for odor analysis, as this requires complex analysis algorithms. QMB technologies have some advantages for certain applications, but the sensitivities are insufficient compared to IMS, DMS, or GC/MS technologies, which may be necessary for many applications. The limit of detection for QBM is in the parts per million range and more recently into the hundreds of parts per billion range.

Another electronic odor sensor embodiment is colorimetric sensors. These sensors may be two-dimensional arrays of chemically active “spots.” Each spot is sensitive to one type of chemical, which is made sensitive by impregnating a disposable cartridge with a chemically sensitive compound that changes color when bound to the analyte to be detected. The chemically sensitive compounds may be metalloporphyrins as well as other materials. The gas is flowed across the sensor, and the changes in color are detected by an optical scanner or camera system, which analyzes and quantifies the detection. These sensors have achieved only moderate success due to the lack of sensitivity and the limited number of spots on the sensor.

Another electronic odor sensor embodiment is conducting polymers. Conductive polymer-based gas sensors are a relatively mature technology and are based on the change in conductance of an organic polymer in the presence of selected analytes. These conductive polymers may be patterned in thin layers over electrodes, which are connected to electronics that sense a change in resistance of the material when exposed to the desired analyte. Scensive Technologies Limited, a company in the United Kingdom started in 1995, has been developing a sensor platform referred to as the Bloodhound, which they claim can detect down to parts per million and parts per billion levels. The Bloodhound and similar e-nose approaches have more difficulty separating out specific analytes, since the sensors in the array are not uniquely sensitive to those compounds and will be confounded by cross-sensitivity to other analytes.

An alternative odor sensor embodiment is surface acoustic wave (SAW) analysis in which compounds are adsorbed onto a thermally controlled piezoelectric crystal. By altering the temperature of the crystal, or by applying a chemically absorptive coating, various compounds can be made to condense or adsorb on the surface of the crystal, thereby providing specificity. In order to simultaneously detect multiple analytes, the crystal is either swept across an appropriate range of temperatures, which is slow, or is duplicated at multiple temperatures, which increases the complexity and cost of the analyzer. The chemically absorptive coating approach limits single devices to detecting only compounds attracted by the coating, and specificity is controlled by the chemistry of the coating-compound interaction.

An alternative analysis configuration is to attach a sampling trap to the debriding tool or patient's skin, and then send the trap to a remote analyzer for odor analysis post de facto. Technically more complex would be an integrated analyzer directly within the tool or on the skin of the patient. Because odors disperse within an air volume, the sampling might be arranged within the treatment room and simply sample room air following interventions, which release the odors to be analyzed.

What is claimed is:

1. A wound care system comprising:
a surgical tool configured for challenging anatomical tissue; and
an electronic odor sensor configured for sensing volatile organic compounds emanating from the anatomical tissue when challenged by the surgical tool.

2. The system as recited in claim 1, further comprising electronics configured for analyzing the volatile organic compounds and producing a feedback signal to a user of the surgical tool.

3. The system as recited in claim 1, wherein the surgical tool is a scalpel.

4. The system as recited in claim 1, wherein the surgical tool is a debridement tool.

5. The system as recited in claim 1, wherein the electronic odor sensor comprises a gas chromatograph coupled to a differential ion mobility spectrometer.

6. The system as recited in claim 5, wherein the electronic odor sensor further comprises an ionizer.

7. The system as recited in claim 6, wherein the ionizer comprises a radioactive ion source.

8. The system as recited in claim 6, wherein the ionizer comprises a non-radioactive ion source.

9. The system as recited in claim 1, wherein the volatile organic compounds comprise bacteria liberated by the surgical tool from underneath a biofilm.
